(19) United States
(12) Patent Application Publication
(10) Pub. No.: US 2003/0059742 A1
(43) Pub. Date: Mar. 27, 2003

(54) Osteointegration device and method

(76) Inventors: Thomas J. Webster, Lafayette, IN (US); Frederick Tepper, Sanford, FL (US)

Correspondence Address: BARNES & THORNBURG, 11 SOUTH MERIDIAN, INDIANAPOLIS, IN, 46204

(21) Appl. No.: 10/253,300

(22) Filed: Sep. 24, 2002

Related U.S. Application Data
(60) Provisional application No. 60/324,398, filed on Sep. 24, 2001.

(51) Int. Cl. A61C 8/00; A61F 2/28

(52) U.S. Cl. 433/201.1; 623/23.51

(57) ABSTRACT

The invention provides an orthopedic/dental implant comprising mineral nanofibers wherein the nanofibers have a diameter of from about 0.1 to about 100 nm. The mineral nanofibers are selected from the group consisting of alumina, titania, zirconia, hafnia, cobalt-chromium, barium aluminate, barium titanate, iron oxide, and zinc oxide nanofibers, or combinations thereof. Also provided is a method of preparation of the implant, a method of treating a patient in need of such an implant, and a method of enhancing osseointegration using the implant.

### CROSS REFERENCE TO RELATED APPLICATIONS

This application claims priority under 35 U.S.C. §119(e) to U.S. Provisional Application No. 60/324,398, filed Sep. 24, 2001.

### FIELD OF THE INVENTION

The present invention relates to orthopedic/dental implants and methods for their preparation and use. More particularly, this invention is directed to orthopedic/dental implants and methods for their preparation and use wherein the orthopedic/dental implants comprise mineral nanofibers having a diameter of from about 0.1 to about 100 nm. The mineral nanofibers are selected from the group consisting of alumina, titania, zirconia, hafnia, cobalt-chromium, barium aluminate, barium titanate, iron oxide, and zinc oxide, or combinations thereof.

### BACKGROUND AND SUMMARY OF THE INVENTION

Sufficient bonding (i.e., osseointegration) between an orthopedic/dental implant and juxtaposed bone is necessary to minimize motion-induced damage to surrounding tissue, to support physiological loading conditions, and the ensure implant efficacy. Insufficient bonding can be linked to implant material surface properties that do not support new bone growth, and/or to mechanical properties that do not match those of surrounding bone tissue. Mismatch in mechanical properties between an implant and surrounding bone can cause stress and strain imbalances leading to crack initiation and eventual implant loosening.

Accordingly, there is a need for orthopedic/dental implants that have surfaces that aid in the formation of new bone to ensure sufficient osseointegration, and, consequently, increased mechanical strength. Osseointegration can be supported by materials that enhance osteoblast (i.e., bone-forming cells) adhesion and proliferation because both of these functions are essential for bone deposition and growth. Thus, orthopedic/dental implant materials are needed that have surface and mechanical properties that enhance osteoblast adhesion and proliferation leading to sufficient osseointegration to juxtaposed bone. In this regard, adhesion of osteoblasts to the implant surface is important, but is not adequate to achieve osseointegration of orthopaedic/dental implants as osteoblast proliferation is also required for osseointegration.

Conventional orthopaedic/dental implant materials, such as commercially pure titanium, Ti-6A1-4V, and CoCrMo alloys, have less than optimal surface properties leading to insufficient osseointegration. Thus, the use of conventional metals and metal alloys can result in implant failure under long-term physiological loading, necessitating the surgical removal of the failed bone implants.

Conventional ceramics, such as hydroxyapatite, bioglasses, bioactive glass ceramics, and calcium phosphate, have also been used in orthopedic/dental implants and are known to enhance formation of new bone. “Conventional” ceramics refers to ceramic grains having a grain size greater than 100 nm. Implants composed of conventional ceramics have also experienced clinical failure. The cause of failure in the case of conventional ceramic implants has been attributed to a lack of direct bonding with bone, that is, insufficient osseointegration resulting in poor mechanical properties, such as low fracture toughness, under physiological loading conditions. Consequently, use of these materials in orthopaedic/dental applications has been limited.

Accordingly, there is a need for orthopedic/dental implant materials with surface properties that aid in the formation of new bone at the tissue-implant interface, and improve orthopedic/dental implant efficacy. The present invention is directed to an orthopedic/dental implant material comprising mineral nanofibers wherein the nanofibers have a diameter of from about 0.1 to about 100 nm. The mineral nanofibers are selected from the group consisting of alumina, titania, zirconia, hafnia, cobalt-chromium, barium aluminate, barium titanate, iron oxide, and zinc oxide, or combinations thereof. The invention is based on the surprising discovery that these nanofiber preparations support enhanced osteoblast adhesion and proliferation which are cellular functions that are essential for bone deposition and growth, and for sufficient osseointegration of orthopedic/dental implants.

In one embodiment an orthopedic/dental implant is provided wherein the implant comprises mineral nanofibers wherein the nanofibers have a diameter of from about 0.1 to about 100 nm

In an alternate embodiment an orthopedic/dental implant is provided wherein the implant comprises nanofibers selected from the group consisting of alumina, titania, zirconia, hafnia, cobalt-chromium, barium aluminate, barium titanate, iron oxide, and zinc oxide nanofibers, or combinations thereof, wherein the nanofibers have a diameter of from about 0.1 to about 100 nm. The implant can further comprise a substrate coated with a composition comprising the nanofibers.

In another embodiment a method of treating a patient in need of an orthopedic/dental implant is provided. The method comprises the steps of selecting the orthopedic/dental implant wherein the implant comprises mineral nanofibers wherein the nanofibers have a diameter of from about 0.1 to about 100 nm and placing the implant into the patient.

In yet another embodiment a method for enhancing osseointegration of an orthopedic/dental implant is provided. The method comprises the steps of selecting the orthopedic/dental implant wherein the implant comprises mineral nanofibers wherein the nanofibers have a diameter of from about 0.1 to about 100 nm and placing the implant into a patient.

In still another embodiment a method of preparing an orthopedic/dental implant is provided. The method comprises the steps of forming a composition comprising mineral nanofibers wherein the nanofibers have a diameter of from about 0.1 to about 100 nm. The method can further comprise the step of coating a substrate with the nanofiber-containing composition. In any of these method embodiments, the nanofibers are selected from the group consisting of alumina, titania, zirconia, hafnia, cobalt-chromium, barium aluminate, barium titanate, iron oxide, and zinc oxide nanofibers, or combinations thereof.

### BRIEF DESCRIPTION OF THE DRAWINGS

FIG. 1 shows a transmission electron micrograph of alumina nanofibers (scale bar=100 nm).

FIG. 2 shows a low magnification scanning electron micrograph of alumina substrates (scale bar=100 μm).

FIG. 3 shows a high magnification scanning electron micrograph of alumina substrates (scale bar=1 μm)

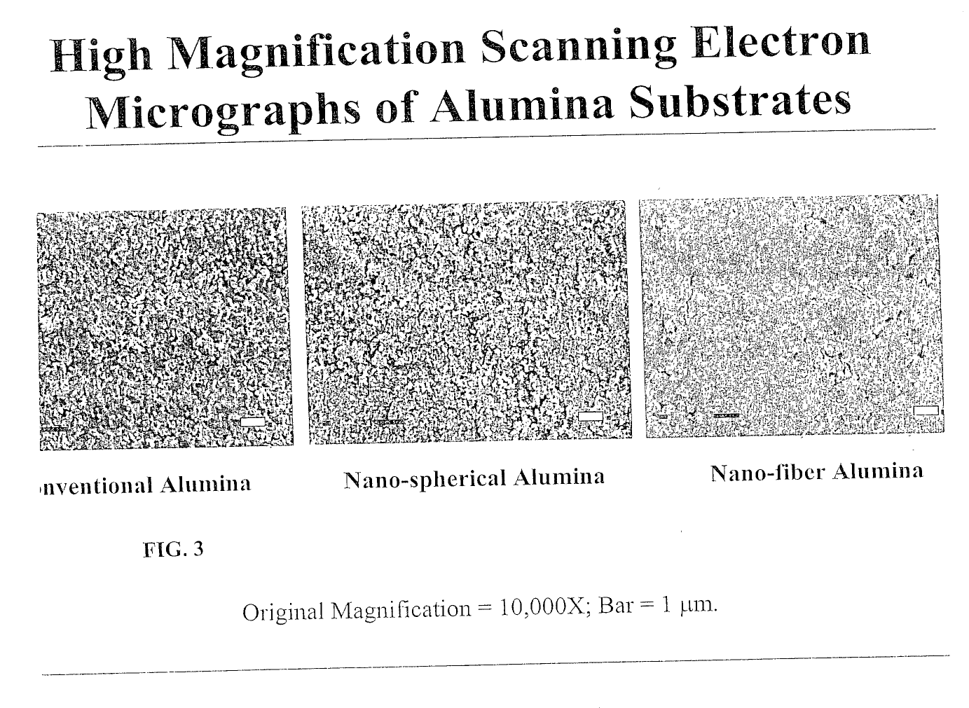

FIG. 4 shows osteoblast adhesion on nanofiber alumina compacts compared to other implant materials.

FIG. 5 shows osteoblast adhesion on alumina nanofibers sintered at various temperatures.

FIG. 6 shows osteoblast proliferation on nanofiber alumina compacts compared to other implant materials.

### DETAILED DESCRIPTION OF THE INVENTION

The present invention is directed to orthopedic/dental implants and methods for their preparation and use wherein the orthopedic/dental implants comprise mineral nanofibers wherein the nanofibers have a diameter of from about 0.1 to about 100 nm. The nanofibers are selected from the group consisting of alumina, titania, zirconia, hafnia, cobalt-chromium, barium aluminate, barium titanate, iron oxide, and zinc oxide nanofibers, or combinations thereof. In alternate embodiments the nanofibers can have a diameter of from about 0.1 to about 70 nm, from about 0.1 to about 40 nm, from about 0.1 to about 30 nm, from about 0.1 to about 20 nm, and from about 0.1 to about 10 nm. Surprisingly, the orthopedic/dental implants in accordance with the present invention support enhanced osteoblast adhesion and proliferation compared to conventional implant materials. The invention may be applicable to a wide range of implants in addition to orthopaedic/dental implants.

The hierarchical organization of the organic and inorganic phases of bone (including porosity and diameters of individual pores) has not been duplicated in formulations that are currently being used as implant materials. In bone tissue collagen and hydroxyapatite fibers are organized parallel to the longitudinal axis of bone resulting in anisotropic mechanical properties with the greatest strength along the principle axis of stress. Moreover, bone is structurally organized into either cancellous (spongy or trabecular) or compact (dense or cortical) bone (Kaplan et al., 1994). Cancellous bone is primarily found at the metaphyses (50% porosity) and epiphyses (90% porosity) of both long and cuboidal bones while compact bone (30% porosity) is primarily found at the diaphyses of long bones and as circular envelopes in cuboidal bone. Cancellous and compact bone have different surface and mechanical properties due to variations in percent porosity and in the diameters of individual surface pores as a result of the organization of constituent fibers.

Bone is a composite material as approximately 30% of its matrix is organic, consisting of 90% collagen and 10% non-collagenous proteins, and 70% is inorganic hydroxyapatite (Kaplan et al., Orthopedic Basic Science (Simon, S. P., ed.) 1994). Furthermore, bone consists of woven and lamellar bone. Woven (i.e., immature) bone has an average inorganic mineral grain size of 10 to 50 nm and is normally found in the metaphysical regions of growing bone as well as in the fracture callus (Kaplan et al., 1994). Lamellar bone actively replaces woven bone and has an average inorganic mineral grain size (e.g., hydroxyapatite) in the shape of fibers 20 to 50 nm long and 2 to 5 nm in diameter (Kaplan et al., 1994). Moreover, in lamellar bone, the inorganic components are highly organized with organic collagen fibers that are nanostructures; these collagen fibers are triple helical bundles with a periodicity of 67 nm as well as length and width of 300 and 0.5 nm, respectively (Ayad et al., The Extracellular Matrix Facts Book, Academic Press, Inc., San Diego, Calif., pp. 29-149, 1994).

Thus, the nanofibers in accordance with the invention have a size in one dimension (i.e., fiber diameter of about 0.1 to about 100 nm) similar to the diameter of hydroxyapatite crystals found in bone. The mineral nanofibers in accordance with the invention can be alumina, titania, zirconia, hafnia, barium aluminate, or barium titanate nanofibers, or any combinations thereof.

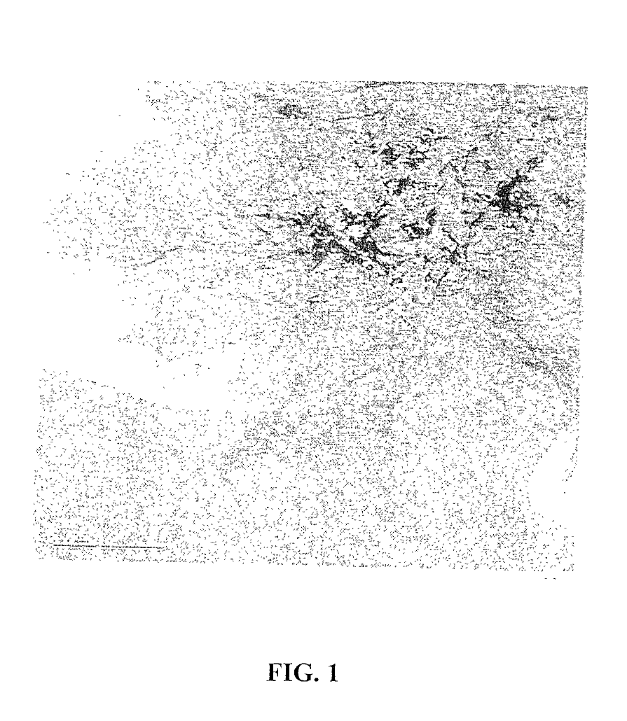

The term “nanofibers” in accordance with the invention means nanosized fibers wherein the diameter of the fibers in one dimension is about 0.1 to about 100 nm. The nanofibers are characterized by a fibrous structure that can be observed by transmission electron microscopy (see FIG. 1, for example).

The process used to produce alumina nanofibers in accordance with the invention is described in detail in Example 1. Alumina nanofibers consist of Al(OH)₃ and boehmite (AlOOH) and some gamma Al₂O₃. Generally, the preparation of the alumina nanofibers for use in accordance with the present invention involves electroexplosion of metal wire in an argon atmosphere to produce a fine metal powder followed by oxidation/hydrolysis of the fine metal powder to make the nanofibers. The process may also require an extra oxidation step following hydrolysis of the metal powder to make the nanofibers, for example, to make titania nanofibers. The procedure described in Example 1 may be applicable to the preparation of any type of mineral nanofiber. The resulting fibers can be about 2 nm in diameter and greater than 50 nm in length, for example, alumina nanofibers can have these approximate dimensions.

As discussed above, bone is a composite matrix material. Thus, the mineral nanofibers in accordance with the invention can be compacted and used alone as implants, or the orthopaedic/dental implant according to the present invention may be a composite material termed herein a “nanocomposite,” incorporating one or more natural or synthetic polymers in addition to the mineral nanofibers. For example, polylactic acid may be combined with the mineral nanofibers to produce a composite having excellent mechanical properties while maintaining the enhanced adhesion of osteoblasts to the nanofibers and the enhanced proliferation exhibited with the mineral nanofibers alone. These nanocomposites may also include an adhesion-promoting peptide as discussed below.

The nanocomposites can comprise about 50-99 parts by weight of a polymer and about 1-50 parts by weight of mineral nanofibers, or can comprise about 30-99

parts by weight of a polymer and about 1-30 parts by weight of mineral nanofibers. The polymer is typically cytocompatible and bioabsorbable and/or bioerodable. It is also non-toxic, non-carcinogenic, and causes no adverse immunologic response. Exemplary polymers include polyfumarates, polylactides, polyglycolides, polycaprolactones, polyanhydrides, pyrollidones, for example, methylpyrollidone and cellulosic polymers, for example, carboxymethyl cellulose, methacrylates, and collagens, for example, gelatin, glycerin and polylactic acid. Synthetic polymer resins may also be used, including, for example, epoxy resins, polycarbonates, silicones, polyesters, polyethers, polyolefins, synthetic rubbers, polyurethanes, nylons, polyvinylaromatics, acrylics, polyamides, polyimides, phenolics, polyvinylhalides, polyphenylene oxide, polyketones and copolymers and blends thereof. Copolymers that can be used include both random and block copolymers. Polyolefin resins that are suitable for use include polybutylene, polypropylene, polyethylene, such as low density polyethylene, medium density polyethylene, high density polyethylene, and ethylene copolymers. Polyvinylhalide resins that can be used include polyvinyl chloride polymers and copolymers, polyvinylidene chloride polymers and copolymers, and fluoropolymers. Polyvinylaromatic resins can also be used and include polystyrene polymers and copolymers and poly.alpha.-methylstyrene polymers and copolymers. Other suitable resins include acrylate resins, such as polymers and copolymers of acrylate, methacrylate esters, polyamide resins, such as nylon 6, nylon 11, and nylon 12, as well as polyamide copolymers and blends thereof, and polyester resins, including polyalkylene terephthalates, such as polyethylene terephthalate and polybutylene terephthalate, as well as polyester copolymers. Synthetic rubbers can be used in combination with the nanofibers in accordance with the invention and include styrene-butadiene and acrylonitrile-butadiene-styrene copolymers and polyketones, including polyetherketones and polyetheretherketones. In one embodiment, the polymer is polylactic acid.

Mineral nanofiber:polymer composites can be formulated according to established procedures (Mikos et al., Polymer 35:1068, 1990). Briefly, various weight concentrations of mineral nanofibers are added to polymer pellets previously dissolved in chloroform, and the nanofiber:polymer composites are then sonicated, vacuum dried, and cured. Polymer dimensions can be controlled by soaking the polymers in either acidic or basic solutions according to established procedures (Gao et al., J. Biomed. Mat. and Res. 42:417, 1998).

To obtain organized layers of mineral nanofibers in polymer matrices, thin (0.5 mm) sheets of polymer and thin (0.5 mm) sheets of mineral nanofibers can be compacted in a tool-steel die via a uniaxial pressing cycle (0.2-1 GPa over a 10 minute period). The orientation of nanofiber:polymer formulations can be controlled by mechanical stretching. For example, orientation of the nanofiber:polymer composite can be controlled by subjecting the composite to tensile stretching to align nanofibers along the principal direction of stress while the composite is being cured. All polymer composites are sterilized by soaking in 70% ethanol for 24 hours prior to use.

To control porosity properties in composite formulations, different weight percentages of salt crystals with various diameters can be added to the viscous nanofiber:polymer composite, and during sonication the salt crystals dissolve leaving pores of desired diameter after soaking in water after curing the polymer (Mikos et al., 1990). The percent porosity of the composites can also be controlled in this manner. Thus, nanofibers could be tailored to meet changes in mechanical properties due to differing porosities of bone associated with either anatomical differences or patient age.

In addition to altering porosity properties it may also be possible to alter the length of the mineral nanofibers. The length of the mineral nanofibers may be altered by varying temperature, pH, ultrasonics, etc. during preparation and compaction of the mineral nanofibers.

An orthopaedic/dental implant according to the present invention may include an adhesion-promoting peptide, if desired. Peptides that promote adhesion between osteoblasts and a substrate, for example, integrin-binding peptides containing the Arginine-Glycine-Aspartic Acid (RGD) sequence are known (Puleo and Bizios, Bone 12: 271-276 (1991)). Published PCT application WO 97/25999, entitled “Peptides for Altering Osteoblast Adhesion,” describes specific peptides, including peptides incorporating the sequence KRSR for enhancement of adhesion to substrates. Adhesion-promoting materials are typically used by attaching the peptide to the surface of a substrate to which adhesion is desired. For example, WO 97/25999 teaches a technique for immobilizing peptides on the surface of a substrate by a silanization reaction. This technique or any other immobilization technique others known in the art may be used to immobilize adhesion-promoting peptides on the surface of implants composed of the mineral nanofibers in accordance with the present invention.

The mineral nanofibers in accordance with the invention can be chemically altered to alter the adsorptive character of the nanofibers as well as fiber size and shape. For example, alkali and alkaline earth metals can be occluded in the mineral nanofiber structure by operating the sol/gel procedure (see Example 1) in the presence of metal salts, such as barium salts, to form combined oxides (e.g., barium aluminate and barium titanate). If occluded in reasonable amounts in mineral nanofibers, barium salts would allow monitoring of the implants to follow the course of osseointegration because of the x-ray opacity of barium salts. Nanofibers comprising hafnia can also form implants with the capacity to be monitored because hafnia is relatively radio-opaque.

Surface derivatization of the mineral nanofibers is also possible and could improve, for example, bonding to polymers, osteoblast adhesion, and osteoblast proliferation. The surface of the nanofibers can be derivatized according to any art-recognized procedures for derivatization of mineral nanofiber materials. For example, the surface of the mineral nanofibers can be reacted with silane compounds to form hydrocarbon chain ligands that can have carboxylic, amine or other functional groups attached. Also, hydroxyl groups can be produced on the mineral nanofibers as a result of either sol/gel precipitation in a mild caustic, or subsequent treatment of the mineral surface with caustic. Surface derivatization of the nanofibers can be used to alter characteristics of the nanofibers such as the dimensions of the nanofibers, nanofiber porosity, and three-dimensional structure of the nanofibers.

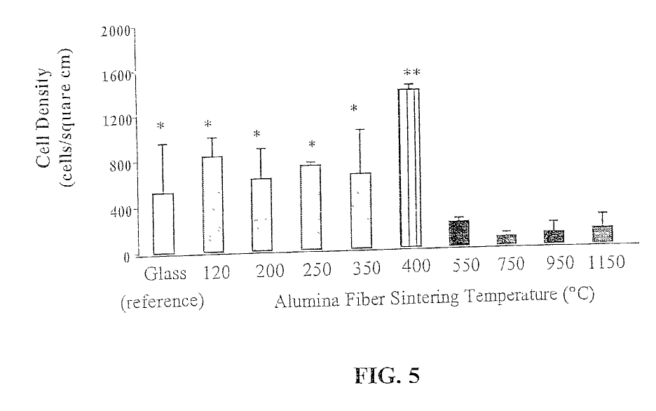

The mineral nanofibers in accordance with the present invention can be sintered to produce porous structures. Sintering may also allow ingress of osteoblasts to improve adhesion and osseointegration. The mineral nanofibers for use in accordance with the invention can be sintered at much lower temperatures (see FIG. 5) than micron size particles. When the mineral nanofibers are heated to about 600° C. or above there is a rapid decline in surface area suggesting that sintering is occurring. In contrast, conventional implant materials, such as conventional alumina, do not sinter until heated to at least 1400° C. The sintered mineral nanofibers wherein the sintered mineral nanofibers are prepared as described in Example 5 are porous structures, but still maintain a fibrous form. In one embodiment the fibers can be sintered in combination with HA particles.

In accordance with the invention the mineral nanofibers are sintered at temperatures ranging from about 100 to about 700° C. In other embodiments the sintering temperature can range from about 200 to about 600° C., from about 300 to about 500° C., from about 350 to about 500° C., from about 350 to about 450° C., or the sintering temperature can be about 400° C. In one embodiment the sintering temperature is about 400° C.

The mineral nanofibers, or nanocomposites thereof with or without adhesion-promoting peptides, can be compacted and/or structured and used alone to form an implant. Alternatively, a structured substrate can be coated with a composition comprising the mineral nanofibers, or nanocomposities thereof with or without adhesion-promoting peptides. Substrates include any conventional substrates for orthopaedic or dental implants or for other types of implants known in the art. Exemplary of such substrates is a substrate comprising titanium metal.

Also provided is a method of treating a patient in need of an orthopedic/dental implant comprising the steps of selecting the orthopedic/dental implant wherein the implant comprises mineral nanofibers wherein the nanofibers have a diameter of from about 0.1 to 100 nm, and placing the implant into the patient. In this embodiment of the invention the term “selecting” means, for example, purchasing, choosing, or providing the implant rather than preparing the implant. The mineral nanofibers are selected from the group consisting of alumina, titania, zirconia, hafnia, cobalt-chromium, barium aluminate, barium titanate, iron oxide, and zinc oxide nanofibers, or combinations thereof. In alternate embodiments the nanofibers can have a diameter of from about 0.1 to about 70 nm, from about 0.1 to about 40 nm, from about 0.1 to about 30 nm, from about 0.1 to about 20 nm, and from about 0.1 to about 10 nm.

The method of the present invention can be used for both human clinical medicine and veterinary applications. Thus, the patient can be a human or, in the case of veterinary applications, can be a laboratory, agricultural, domestic, or wild animal. The present invention can be applied to animals including, but not limited to, humans, laboratory animals such as monkeys and chimpanzees, domestic animals such as dogs and cats, agricultural animals such as cows, horses, pigs, sheep, goats, and wild animals in captivity such as bears, pandas, lions, tigers, leopards, elephants, zebras, giraffes, gorillas, dolphins, and whales.

In another embodiment a method for enhancing osseointegration of an orthopedic/dental implant is provided. The method comprises the steps of selecting the orthopedic/dental implant wherein the implant comprises mineral nanofibers wherein the nanofibers have a diameter of about 0.1 to about 100 nm, and placing the implant into a patient. In this embodiment of the invention the term “selecting” means, for example, purchasing, choosing, or providing the implant rather than preparing the implant. The mineral nanofibers are selected from the group consisting of alumina, titania, zirconia, hafnia, cobalt-chromium, barium aluminate, barium titanate, iron oxide, and zinc oxide nanofibers, or combinations thereof. In alternate embodiments the nanofibers can have a diameter of from about 0.1 to about 70 nm, from about 0.1 to about 40 nm, from about 0.1 to about 30 nm, from about 0.1 to about 20 nm, and from about 0.1 to about 10 nm. The patient can be a human or, in the case of veterinary applications, can be a laboratory, agricultural, domestic, or wild animal.

Enhancement of osseointegration is increased osseointegration compared to that obtained with conventional implant materials, including conventional titania and alumina implants, hydroxyapatite, and nanospherical alumina. Enhanced osseointegration can be demonstrated by increased osteoblast adhesion, increased osteoblast proliferation, increased calcium deposition, enzyme activity assays, or by any other art-recognized technique used to detect osseointegration.

In yet another embodiment a method of preparing an orthopedic/dental implant is provided. The method comprises the step of forming a composition comprising mineral nanofibers wherein the nanofibers have a diameter of from about 0.1 to about 100 nm. The method can further comprise the step of coating a substrate with the nanofiber-containing composition. The mineral nanofibers are selected from the group consisting of alumina, titania, zirconia, hafnia, cobalt-chromium, barium aluminate, barium titanate, iron oxide, and zinc oxide nanofibers, or combinations thereof. In alternate embodiments the nanofibers can have a diameter of from about 0.1 to about 70 nm, from about 0.1 to about 40 nm, from about 0.1 to about 30 nm, from about 0.1 to about 20 nm, and from about 0.1 to about 10 nm.

The mineral nanofiber composition formed can be a composition containing the mineral nanofibers alone, a nanocomposite composition, a nanocomposite composition containing an adhesion-promoting peptide, or any other composition containing mineral nanofibers that is suitable for use in accordance with the present invention. The mineral nanofiber composition can be compacted and/or structured or a structured substrate can be coated with the composition comprising the mineral nanofibers.

### EXAMPLE 1

### Preparation of Alumina Nanofibers

Alumina nanofibers were prepared by using nano aluminum powder (i.e., grains with a diameter of approximately 100 nm). The process used to produce the nano aluminum powders involves electroexplosion of metal wire in an argon atmosphere. The nano aluminum powder used is produced by Argonide Corporation (Sanford, Fla.) and is sold under the tradename Alex®.

Briefly, the powder is produced by enclosing a reel of 0.3 mm diameter aluminum wire in an argon filled chamber at a 3 atmosphere pressure and the wire is then fed through an electrically insulated baffle into a separate region of the chamber. When the wire contacts a strike plate located in the center of the chamber, the circuit is closed causing a large pulse (10²-10³ joules during a microsecond) from a capacitor bank to flow through the wire, creating a metal plasma. A very strong field, created during the microsecond pulse, contains the plasma. When the vapor pressure of the metal exceeds the force of the field there is an interruption in current flow. This interruption in current flow causes the plasma to explode into clusters of metal that are then projected at supersonic speeds (˜2 km/sec) through the argon, quenching the metal. The process is semi-continuous in that wire is constantly fed into a reactor and the current procedure involves manual removal of approximately 0.5 to 1 kg of powder from the collector.

The nano aluminum powder particles are spherical, fully dense and typically about 100 nanometers in size. In the collector, there is further coalescence of the spherical particles into agglomerates that are about 2-5 microns across. Before removal from the collectors, the powders are exposed to argon containing dry air so as to slowly passivate the powder to form an aluminum oxide coating about 3 nm thick to prevent the particles from being pyrophoric. X-ray diffraction shows the powders to be aluminum (90-92%) with minor phases of aluminum oxide, aluminum nitride, and a trace of an oxynitride.

The conversion of the nano aluminum powder into ceramic fibers was accomplished by digestion of the powder in water at room temperature. Five kilograms of the powder was put into a stainless steel reactor about 50 cm in diameter and about 40 cm high and water was added at 75° C. Hydrogen was generated and vented from the top of the reactor. A sol of aluminum hydroxide Al(OH)₃ was formed, and the sol was discharged through a filter, collected, and dried at approximately 100° C. The resulting fibers were then heated in a recirculating air oven at 200-450° C. for approximately 4 hours. A substantial amount of excess and chemically combined water was displaced from the fibers. The resulting fibers consist of boehmite (AlOOH) and Al(OH)₃ and some gamma Al₂O₃. If the temperature is increased, the yield of boehmite increases, and if the temperature is lowered, the yield of tri-hydroxide increases. The resulting fibers are about 2 nm in diameter and greater than 50 nm in length.

Although 100 nm diameter nano aluminum powder was used for this procedure, any type of nano size or micron size (e.g., 5 micron diameter aluminum powder can be used to produce the fibers) aluminum powder can be used. The above-described process can also be modified to produce nano size oxides such as alumina or nitrides such as AlN.

An alternate method involves electro-exploding aluminum wire in a nitrogen environment at 3 atmospheres absolute pressure. If nitrogen is used, the passivation step can be eliminated since the nitride coated nano aluminum is not pyrophoric. In this case, the aluminum metal particle is coated with a layer of aluminum nitride (AlN), and when hydrolyzed, boehmite fibers are produced.

### EXAMPLE 2

### Transmission Electron Micrograph of Alumina Nanofibers

FIG. 1 shows a transmission electron micrograph of the alumina nanofibers produced by the above-described process (scale bar=100 nm). The measured surface area (BET) of the fiber is dependent upon final heat treatment, with the peak being greater than 600 m²/g when heat-treated to 250° C. Computation of external surface area for a 2 nm fiber gives an external surface area about 500 m²/g, suggesting that much of the measured surface area is external rather than in internal pores. The fibers are co-mingled in a wooly structure, and areas of opacity do not appear to be agglomerates, but are artifacts of viewing several fibers in the field. X-ray diffraction and Fourier Transform Infra-Red Spectra show the fibers to be principally aluminum hydroxide and boehmite (AlOOH).

### EXAMPLE 3

### Scanning electron Micrographs of Alumina nanofibers

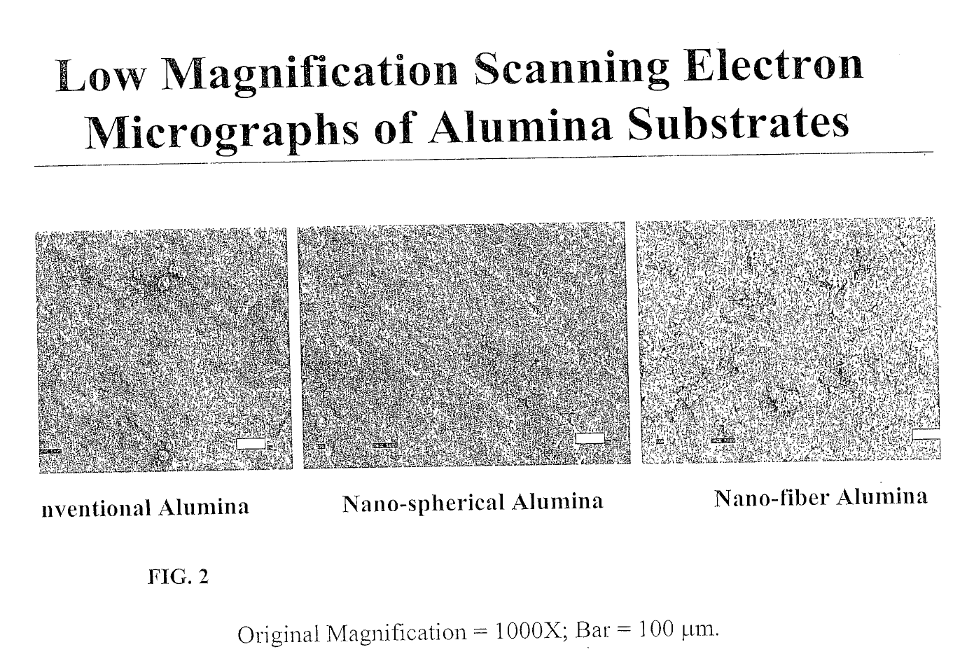

Conventional alumina, nanospherical alumina, and alumina nanofiber substrates were examined by scanning electron microscopy (JOEL JSM-840 scanning electron microscope) for surface properties, specifically topography and roughness. Samples were sputter-coated in gold at room temperature. Representative images are shown in FIGS. 2 (scale bar=100 μM) and 3 (scale bar=1 μM).

### EXAMPLE 4

### Enhanced Osteoblast Adhesion on Nanofiber Alumina

For the present studies, compacts of the above-described alumina fibers with dimensions in the nanometer range were prepared and tested for cytocompatibility properties. To prepare the compacts, the fibers as shown in FIG. 1 were compacted serially in a tool-steel die via a uniaxial pressing cycle (0.2-1 GPa over a 10 minute period), sterilized in an autoclave at 120° C. for 35 minutes, followed by drying in a 130° C. oven for 20 minutes, and then used immediately in cell experiments. The resulting alumina nanofiber compacts were 10 mm in diameter and 2 mm thick.

Conventional orthopedic/dental implant materials, such as commercially available borosilicate glass, pure titanium, micron grain size synthetic hydroxyapatite, as well as spherical micron and nanometer grain size alumina substrates were also prepared for comparative purposes. Briefly, borosilicate glass coverslips were etched in 1 N NaOH and were prepared for experiments according to standard protocols. Pure titanium is commercially available (Osteonics, Allendale, N.J.).

Nanophase alumina powder (23 nm spherical alumina particle size; gamma-phase; Nanophase Technologies Corp., Romeoville, Ill.) was compacted at room temperature as described above for alumina nanofibers. Alumina grain size was controlled to less than 100 nm by heating (in air at 10° C./minute) the compacts from room temperature to a final temperature of 1000° C. and by sintering at 1200° C. for two hours.

Conventional substrates with grain sizes greater than 100 nm were obtained by heating (in air at 10° C./minute) alumina compacts from room temperature to a final temperature of 1200° C. and by sintering at 1200° C. for two hours. Both nanophase and conventional spherical grain size compacts prepared in this manner were gamma-phase alumina and not a hydrated from as are the alumina nanofibers.

Hydroxyapatite (HA) was synthesized by dripping 1 M calcium nitrate and 0.6 M ammonium phosphate into a solution of distilled water and ammonium hydroxide. The solution was stirred for 24 hours at room temperature, while HA precipitated out. The solution was centrifuged and rinsed in triplicate, then dried for 24 hours in a glass-drying oven (70° C.). The resulting powder was crushed, pressed into a compact (10 mm in diameter and 2 mm thick) via a uniaxial pressing cycle (0.2-1 GPa over a 10 minute period), and sintered at 1100° C. for 1 hour. All material samples were degreased, ultrasonically cleaned, and sterilized in a steam autoclave at 120° C. for 30 minutes followed by drying in a 130° C. oven for 20 minutes according to standard laboratory procedures prior to experiments with osteoblasts.

For adhesion experiments, human osteoblasts (CRL-11372; ATCC) in Dulbecco's Modified Eagle Medium (Gibco) supplemented with 10% fetal bovine serum (Gibco) were seeded at a density of 2,500 cells/cm² onto the substrates of interest, and were allowed to adhere under standard cell culture conditions (a humidified, 37° C., 5% CO₂/95% air environment) for two hours. At that time, nonadherent cells were removed by rinsing in phosphate buffered saline while adherent cells were fixed with formalin, stained with Hoeschst 33258 dye (Sigma), and counted under a fluorescence microscope (Kim et al., Anal. Biochem. 174:168, 1988). Cell counts were expressed as the average number of five random fields per square cm of substrate surface area.

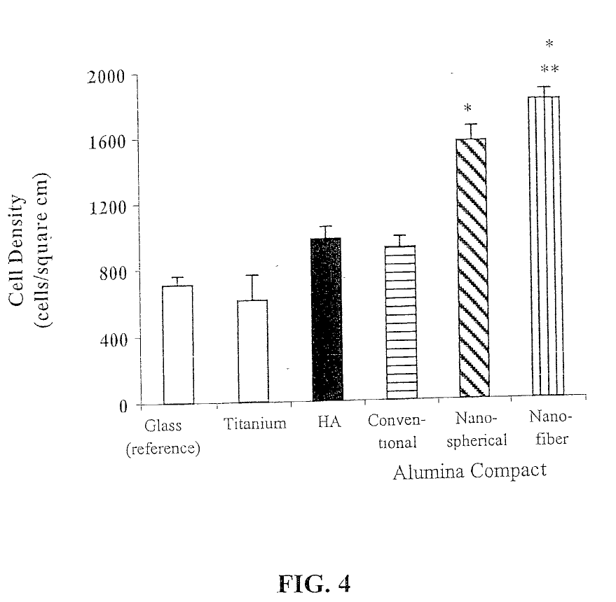

The results (see FIG. 4) provided evidence of significantly (p<0.01) greater osteoblast adhesion on the alumina nanofibers compared to commercially pure titanium (Osteonics), hydroxyapatite, conventional alumina (micron size), and nanospherical alumina. Values are mean±SEM; * p<0.01 (compared to titanium); ** p<0.05 (compared to nanospherical alumina).

All cell experiments (adhesion and proliferation) were run in triplicate and repeated a minimum of three times per substrate type. Numerical data was analyzed using Analysis of Variance (ANOVA) techniques with factorial designs; values of p<0.05 are considered significant differences between substrate types (Montgomery, Design and Analysis of Experiments, John Wiley and Sons, N.Y., 1991).

### EXAMPLE 5

### Enhanced Osteoblast Adhesion on Low Temperature Sintered Alumina Nanofibers

The procedures were similar to those described in Example 4 except that the alumina nanofibers were sintered at various temperatures before compaction. As shown in FIG. 5, osteoblast adhesion was significantly greater on alumina nanofibers that were sintered (before compaction) at or near 400° C. There was considerable fall-off in adhesion (p<0.1) for those samples heat-treated at or above 550° C. or at or below 350° C. Values are mean±SEM; * p<0.1; ** p<0.01 (compared to alumina sintered at 555° C.).

### EXAMPLE 6

### Enhanced Osteoblast Proliferation of Alumina Nanofiber

For proliferation experiments, osteoblasts were seeded at a density of 2,500 cells/cm² onto the substrates of interest, and were cultured for 1, 3, and 5 days in a similar manner as described above for the adhesion experiments. At the conclusion of the experiments, cells were fixed, stained, and counted as described above. Values are mean±SEM; * p<0.01 (compared to titanium); ** p<0.01 (compared to nanospherical alumina).

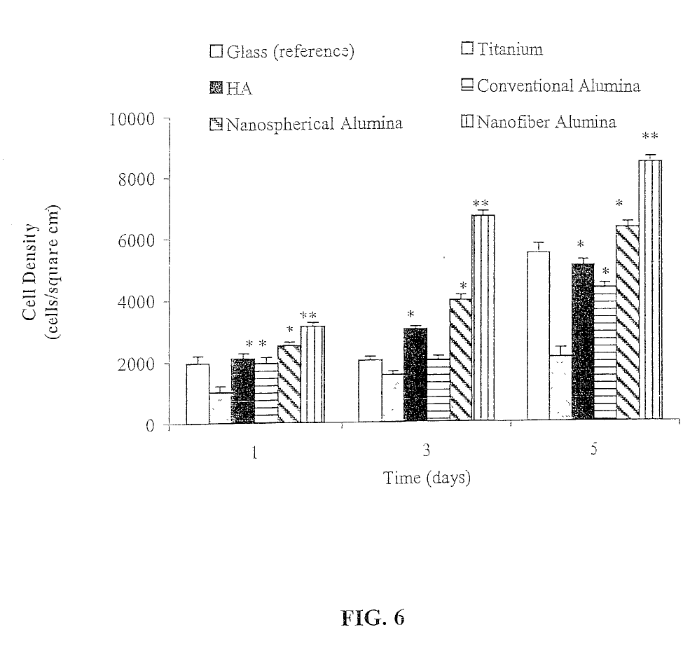

The above-described results provide the first evidence of increased osteoblast adhesion (FIG. 4) and proliferation (FIG. 6) on the novel alumina nanofiber substrates compared to all other substrates tested.

What is claimed is:

1. An orthopedic/dental implant comprising mineral nanofibers wherein the nanofibers have a diameter of from about 0.1 to about 100 nm.

2. The implant of claim 1 wherein the nanofibers have a diameter of from about 0.1 to about 70 nm.

3. The implant of claim 1 wherein the nanofibers have a diameter of from about 0.1 to about 30 nm.

4. The implant of claim 1 wherein the nanofibers have a diameter of from about 0.1 to about 20 nm.

5. The implant of claim 1 wherein the nanofibers have a diameter of from about 0.1 to about 10 nm.

6. The implant of claim 1 comprising a substrate coated with a composition comprising the nanofibers.

7. The implant of claim 1 further comprising an adhesion-promoting peptide.

8. The implant of claim 7 wherein the peptide comprises a KRSR or an RGD sequence.

9. The implant of claim 1 further comprising a non-peptide polymer.

10. The implant of claim 9 wherein the polymer is polylactic acid.

11. The implant of claim 1 wherein the nanofibers are sintered.

12. The implant of claim 11 wherein the sintering temperature ranges from about 100 to about 700° C.

13. The implant of claim 11 wherein the sintering temperature ranges from about 200 to about 600° C.

14. The implant of claim 11 wherein the sintering temperature ranges from about 300 to about 500° C.

15. The implant of claim 11 wherein the sintering temperature ranges from about 350 to about 500° C.

16. The implant of claim 11 wherein the sintering temperature ranges from about 350 to about 450° C.

17. The implant of claim 11 wherein the sintering temperature is 400° C.

18. The implant of claim 1 wherein the mineral nanofibers are alumina nanofibers.

19. The implant of claim 18 wherein the alumina nano fibers further comprise boehmite.

20. An orthopedic/dental implant comprising mineral nanofibers selected from the group consisting of alumina, titania, zirconia, hafnia, cobalt-chromium, barium aluminate, barium titanate, iron oxide and zinc oxide nanofibers, or combinations thereof, wherein the nanofibers have a diameter of from about 0.1 to about 100 nm.

21. A method of treating a patient in need of an orthopedic/dental implant comprising the steps of
selecting the orthopedic/dental implant wherein the implant comprises mineral nanofibers wherein the nanofibers have a diameter of from about 0.1 to 100 nm; and
placing the implant into the patient.

22. The method of claim 21 wherein the mineral nanofibers are selected from the group consisting of alumina, titania, zirconia, hafnia, cobalt chromium, barium aluminate, barium titanate, iron oxide, and zinc oxide nanofibers, or combinations thereof.

23. A method for enhancing osseointegration of an orthopedic/dental implant comprising the steps of
selecting the orthopedic/dental implant wherein the implant comprises mineral nanofibers wherein the nanofibers have a diameter of about 0.1 to about 100 nm; and
placing the implant into a patient.

24. The method of claim 23 wherein the mineral nanofibers are selected from the group consisting of alumina, titania, zirconia, hafnia, cobalt chromium, barium aluminate, barium titanate, iron oxide, and zinc oxide nanofibers, or combinations thereof.

25. A method of preparing an orthopedic/dental implant comprising the step of forming a composition comprising mineral nanofibers wherein the nanofibers have a diameter of from about 0.1 to about 100 nm.

26. The method of claim 25 further comprising the step of coating a substrate with the nanofiber-containing composition.

27. The method of claim 25 wherein the mineral nanofibers are selected from the group consisting of alumina, titania, zirconia, hafnia, cobalt-chromium, barium aluminate, barium titanate, iron oxide, and zinc oxide nanofibers, or combinations thereof.
